(19) United States
(12) Patent Application Publication
(10) Pub. No.: US 2026/0130831 A1
(43) Pub. Date: May 14, 2026

(54) HAIR STYLING PRODUCT IN PELLET FORM

(71) Applicant: Jeffrey S. Goe, Largo, FL (US)

(72) Inventor: Jeffrey S. Goe, Largo, FL (US)

(21) Appl. No.: 18/933,972

(22) Filed: Nov. 14, 2024

(51) Int. Cl. A61K 8/02; A61Q 5/06

(52) U.S. Cl. CPC A61K 8/0216; A61Q 5/06; A61K 2800/87

(57) ABSTRACT

A hair styling product in pellet form is disclosed. The product comprises a plurality of pellets, each containing a pre-measured amount of hair styling composition. The pellets are designed to melt or soften when exposed to body heat, allowing for easy application to the hair. The hair styling composition may include organic, natural ingredients. The pellets may be stored in a container designed to maintain product freshness and allow easy access to individual pellets. Methods of applying the hair styling product and systems for hair styling using the pellets are also disclosed. The product provides advantages in terms of precise measurement, sanitary application, and convenience of use.

### FIELD OF THE INVENTION

The present disclosure relates to the field of personal care products, particularly hair styling aids. More specifically, it pertains to hair pomades and clay-based styling products in the form of individual, pre-measured pellets that are heat-activated for easy application.

### BACKGROUND

Hair styling products, such as pomades and clays, are commonly used to style, shape, and hold hair in a desired configuration. Traditionally, these products have been provided in the form of creams or pastes contained in jars or tubs. Users typically apply these products by scooping a portion from the container with their fingers and then spreading it onto their hands before applying it to their hair.

This traditional method of application may present several drawbacks. For example, it may be difficult for users to consistently measure the appropriate amount of product for each application, potentially resulting in the use of too much or too little product. Additionally, repeatedly dipping fingers into the product container may introduce contaminants, potentially leading to bacterial growth and compromising the quality and safety of the product over time.

There exists a need for an improved hair styling product that addresses these and other limitations of traditional hair styling products.

### SUMMARY

The present disclosure relates generally to hair styling products, and more particularly to a hair styling product in pellet form. The present disclosure provides a hair styling product in pellet form and methods for its use. In various embodiments, the hair styling product comprises a plurality of individual pellets, each pellet containing a pre-measured amount of hair styling composition. The pellets may be stored in a container and may be designed to melt or soften when exposed to body heat, allowing for easy application to the hair.

In one aspect, a hair styling product is provided. The hair styling product may comprise a plurality of pellets, each pellet comprising a hair styling composition. The hair styling composition may include one or more organic, natural ingredients. The pellets may be in any shape or form.

In another aspect, a method of applying a hair styling product is provided. The method may comprise removing a pellet from a container, placing the pellet in a user's palm, applying pressure and heat to the pellet to melt or soften it, spreading the melted or softened product across both palms, and applying the product to the hair or beard.

In yet another aspect, a system for hair styling is provided. The system may comprise a container and a plurality of hair styling pellets stored within the container. The container may be designed to allow easy access to individual pellets while maintaining the freshness and integrity of the remaining pellets. These and other features, aspects, and advantages of the present disclosure will become better understood with reference to the following description and appended claims.

These and other features, aspects, and advantages of the present disclosure will become better understood with reference to the following description and appended claims. The accompanying drawings, which are incorporated in and constitute a part of this specification, illustrate embodiments of the disclosure and, together with the description, serve to explain the principles of the disclosure. Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF DRAWINGS

The features and advantages of the present disclosure will become more apparent from the detailed description set forth below when taken in conjunction with the drawings. The accompanying drawings, which are incorporated in and constitute a part of this specification, illustrate several aspects of the invention and together with the description, serve to explain the principles of the invention.

FIG. 1 illustrates an exemplary embodiment of containers holding hair styling pellets in accordance with the present disclosure.

The features and advantages of the present disclosure will become more apparent from the detailed description set forth below when taken in conjunction with the drawings. The accompanying drawings, which are incorporated in and constitute a part of this specification, illustrate several aspects of the invention and together with the description, serve to explain the principles of the invention.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts. The present invention can be understood more readily by reference to the following detailed description of the invention and the examples included therein. Before the present articles, systems, devices, and/or methods are disclosed and described, it is to be understood that they are not limited to specific manufacturing methods unless otherwise specified, or to particular materials unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described. All publications mentioned herein are incorporated herein by reference to disclose and describe the methods and/or materials in connection with which the publications are cited.

### Definitions

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this present disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an opening” can include two or more openings.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Disclosed are the components to be used to manufacture the disclosed devices, systems, and articles of the present disclosure as well as the devices themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the present disclosure. Thus, if there are a variety of additional steps that can be performed it is understood that each of these additional steps can be performed with any specific aspect or combination of aspects of the methods of the present disclosure.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result. All publications mentioned herein are incorporated herein by reference to disclose and describe the methods and/or materials in connection with which the publications are cited.

The present disclosure provides a hair styling product in pellet form and methods for its use. While the present disclosure is susceptible to various modifications and alternative forms, specific embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the disclosure as defined by the appended claims.

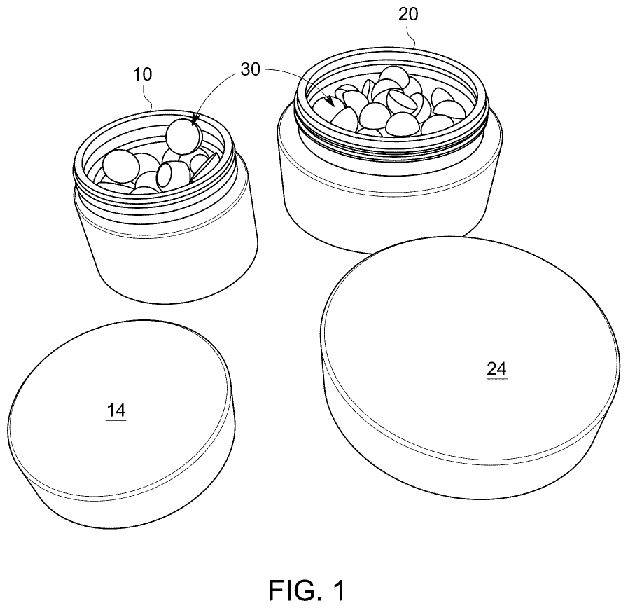

Referring now to FIG. 1, an exemplary embodiment of containers holding hair styling pellets is illustrated. The figure shows two open containers 10, 20, one black container 10 and one clear container 20, filled with small, round pellets 30. The black container 10 has a black base, and the clear container 20 has a frosted base. Both containers 10, 20 have similar, light-colored wooden lids 14, 24 placed beside them on a wooden surface 40.

In various embodiments, the hair styling product of the present disclosure may comprise a plurality of individual pellets 30, each pellet containing a pre-measured amount of hair styling composition. The pellets 30 may be substantially spherical, oval, or pill-shaped, and may have a size similar to that of a typical pill or small candy. The exact size and shape of the pellets 30 may vary depending on factors such as the desired amount of product per application, the specific formulation of the hair styling composition, and user preferences.

The hair styling composition used to form the pellets 30 may comprise one or more organic, natural ingredients. These ingredients may be selected to provide desired styling properties such as hold, texture, shine, or matte finish. The specific formulation may vary depending on the intended use and desired characteristics of the final product. For example, different formulations may be used to create pomades, clays, or other types of styling products.

In some embodiments, the hair styling composition may include ingredients that promote hair growth or provide other beneficial effects to the hair or scalp. The composition may be formulated to be free from harsh chemicals, synthetic fragrances, or other potentially harmful ingredients.

The pellets 30 may be designed to melt or soften when exposed to body heat, such as the warmth of a user's hands. This property allows the solid pellet to transform into a spreadable styling product upon application, providing ease of use and even distribution of the product.

The containers 10, 20 used to store the pellets 30 may be designed to allow easy access to individual pellets while maintaining the freshness and integrity of the remaining pellets. The containers 10, 20 may be airtight to prevent contamination and preserve the quality of the product. In some embodiments, the containers 10, 20 may include a dispensing mechanism to facilitate the removal of individual pellets.

A method of applying the hair styling product may comprise the following steps:
a. Opening the container 10, 20.
b. Removing a single pellet 30 from the container 10, 20.
c. Placing the pellet 30 in the palm of the hand.
d. Applying pressure and heat to the pellet 30 to melt or soften it.
e. Spreading the melted or softened product evenly across both palms.
f. Applying the product to the hair or beard for styling.
g. Closing the container 10, 20 to preserve the remaining pellets 30.

This method of application provides several advantages over traditional hair styling products. First, it allows for precise, pre-measured amounts of product to be used for each application, reducing waste and ensuring consistent results. Second, it minimizes the risk of contamination, as users do not need to repeatedly dip their fingers into a shared product container. Finally, the pellet form factor may make the product more portable and convenient for travel or on-the-go use.

The hair styling product of the present disclosure may be used for various hair types and styles. It may be suitable for use on both hair and beards, and may be used by individuals of any gender. Different sizes or formulations of pellets may be provided for different hair lengths, beard styles, or specific styling needs such as frizz control.

The hair styling product of the present disclosure may comprise a plurality of individual portions, each portion containing a pre-measured amount of hair styling composition. The individual portions may be formed into various shapes, such as pellets, tablets, discs, cubes, or other geometric forms. The exact shape of the individual portions

may vary depending on factors such as manufacturing considerations, user preferences, and ease of handling.

In some embodiments, the individual portions may be substantially spherical, forming small balls or beads. In other embodiments, the portions may be flattened into disc or coin-like shapes. Alternatively, the portions may be formed into small cubes or rectangular prisms. The shape may be selected to optimize factors such as ease of grasping a single portion, rate of melting when exposed to body heat, and aesthetics of the product.

Regardless of the specific shape chosen, each individual portion may be designed to contain a consistent, pre-measured amount of the hair styling composition. This pre-measured portioning may allow users to easily select the appropriate amount of product for their styling needs without the guesswork associated with traditional cream or paste formulations dispensed from jars.

The size of each individual portion may be tailored to provide an amount of product suitable for a single application. For example, a typical portion may be sized similarly to a small pill or candy, making it easy to pick up and handle. The exact dimensions may vary based on factors such as the density of the composition, the typical amount needed for styling, and user preferences.

In various embodiments, different sizes of individual portions may be provided to accommodate different hair lengths, styling needs, or application areas. For instance, larger portions may be offered for longer hair or full-head styling, while smaller portions may be available for touch-ups, short hair, or beard styling.

The hair styling composition used to form the individual portions may comprise one or more organic, natural ingredients. These ingredients may be selected to provide desired styling properties such as hold, texture, shine, or matte finish. The specific formulation may vary depending on the intended use and desired characteristics of the final product.

The individual portions may be designed to melt or soften when exposed to body heat, such as the warmth of a user's hands. This property allows the solid portion to transform into a spreadable styling product upon application, providing ease of use and even distribution of the product.

A container may be provided to store the plurality of individual portions. The container may be designed to allow easy access to individual portions while maintaining the freshness and integrity of the remaining portions. The container may be airtight to prevent contamination and preserve the quality of the product. In some embodiments, the container may include a dispensing mechanism to facilitate the removal of individual portions.

The method of application may provide several advantages over traditional hair styling products. First, it may allow for precise, pre-measured amounts of product to be used for each application, reducing waste and ensuring consistent results. Second, it may minimize the risk of contamination, as users do not need to repeatedly dip their fingers into a shared product container. Finally, the individual portion form factor may make the product more portable and convenient for travel or on-the-go use.

The hair styling product of the present disclosure may be used for various hair types and styles. It may be suitable for use on both hair and beards, and may be used by individuals of any gender. Different sizes or formulations of individual portions may be provided for different hair lengths, beard styles, or specific styling needs such as frizz control.

In some embodiments, the hair styling composition may include ingredients that promote hair growth or provide other beneficial effects to the hair or scalp. The composition may be formulated to be free from harsh chemicals, synthetic fragrances, or other potentially harmful ingredients.

The individual portioning concept described herein may potentially be applied to other personal care products beyond hair styling. For example, similar individual portions could be developed for face moisturizers, body lotions, or other cosmetic products where precise dosing and sanitary application are desirable.

While the present disclosure has been described with reference to specific embodiments, it should be understood that various changes may be made and equivalents may be substituted for elements thereof without departing from the true spirit and scope of the disclosure. In addition, many modifications may be made to adapt a particular situation or material to the teachings of the disclosure without departing from its essential scope.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The present disclosure is to be limited only by the terms of the appended claims, along with the full scope of equivalents to which such claims are entitled. It is to be understood that this disclosure is not limited to particular methods, reagents, compounds compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications can be referenced. The disclosures of these publications in their entirety are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein can be different from the actual publication dates, which can require independent confirmation.

The patentable scope of the invention is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

Although very narrow claims are presented herein, it should be recognized the scope of this disclosure is much broader than presented by the claims. It is intended that broader claims will be submitted in an application that claims the benefit of priority from this application. It should be understood that the above-described embodiments are merely possible examples of implementations, merely set forth for a clear understanding of the principles of the present disclosure. Many variations and modifications may be made to the above-described embodiments without departing substantially from the spirit and principles of the disclosure. All such modifications and variations are intended to be included herein within the scope of this disclosure and protected by the following claims.

What is claimed is:

1. A hair styling product comprising:
a plurality of pellets, each pellet comprising a hair styling composition;
wherein each pellet is configured to melt or soften when exposed to body heat; and
wherein the hair styling composition comprises one or more organic, natural ingredients.

2. The hair styling product of claim 1, wherein each pellet is substantially spherical, oval, or pill-shaped.

3. The hair styling product of claim 1, wherein each pellet contains a pre-measured amount of the hair styling composition.

4. The hair styling product of claim 1, further comprising a container configured to store the plurality of pellets.

5. The hair styling product of claim 4, wherein the container is airtight.

6. The hair styling product of claim 4, wherein the container comprises a dispensing mechanism configured to facilitate removal of individual pellets.

7. The hair styling product of claim 1, wherein the hair styling composition is formulated to promote hair growth.

8. The hair styling product of claim 1, wherein the hair styling composition is free from synthetic fragrances.

9. The hair styling product of claim 1, wherein the plurality of pellets comprises pellets of different sizes for different hair lengths or styling needs.

10. A method of applying a hair styling product, the method comprising:
removing a pellet from a container, the pellet comprising a hair styling composition;
placing the pellet in a user's palm;
applying pressure and heat to the pellet to melt or soften it;
spreading the melted or softened product across both palms; and
applying the product to hair or beard.

11. The method of claim 10, further comprising closing the container after removing the pellet to preserve remaining pellets.

12. The method of claim 10, wherein the hair styling composition comprises one or more organic, natural ingredients.

13. The method of claim 10, wherein the pellet contains a pre-measured amount of the hair styling composition.

14. The method of claim 10, wherein the container is airtight.

15. The method of claim 10, wherein removing the pellet comprises using a dispensing mechanism of the container.

16. A system for hair styling, the system comprising:
a container; and
a plurality of hair styling pellets stored within the container;
wherein each pellet comprises a hair styling composition configured to melt or soften when exposed to body heat.

17. The system of claim 16, wherein the container is configured to allow easy access to individual pellets while maintaining freshness and integrity of remaining pellets.

18. The system of claim 16, wherein the container is airtight.

19. The system of claim 16, wherein the container comprises a dispensing mechanism configured to facilitate removal of individual pellets.

20. The system of claim 16, wherein the hair styling composition comprises one or more organic, natural ingredients.
